(19) United States
(12) Patent Application Publication
(10) Pub. No.: US 2011/0295238 A1
(43) Pub. Date: Dec. 1, 2011

(54) DEVICE FOR FLUID JET-SUPPORTED SEPARATION AND SUCTIONING OF TISSUE CELLS FROM A BIOLOGICAL STRUCTURE

(75) Inventors: ARND KENSY, MICHENDORF (DE); KONRAD-WENZEL WINKLER, WARIN (DE)

(73) Assignee: HUMAN MED AG, SCHWERIN (DE)

(21) Appl. No.: 13/108,400

(22) Filed: May 16, 2011

Related U.S. Application Data
(60) Provisional application No. 61/348,308, filed on May 26, 2010.

(51) Int. Cl. A61M 1/00

(52) U.S. Cl. 604/542

(57) ABSTRACT

The invention relates to a device for fluid jet-supported separation and suctioning of tissue cells from a biological structure, with a pressure generator for supplying a defined fluid jet to an applicator of the device, with a suction device for removing the separated tissue cells and the used fluid from the biological structure, wherein the suction device includes a vacuum source and a suction line for connecting the applicator with the vacuum source, wherein a tissue cell collector is incorporated in the suction line.

It is provided that a catch device (16) for catching connecting tissue and the like from the fluid-tissue cell mixture is arranged upstream of the tissue cell collector (13).

The invention relates to a device for fluid jet-supported separation and suctioning of tissue cells from a biological structure, a with a pressure generator for supplying a defined fluid jet to an applicator of the device, with a suction device for removing the separated tissue cells and the used fluid from the biological structure, wherein a suction device includes a vacuum source and a suction line for connecting the applicator with the vacuum source, wherein a tissue cell collector is incorporated in the suction line.

A device of the generic type is known, for example, from WO 2009/149691 A2. Described herein is a device for separating tissue cells from a fluid, consisting of a tissue cell collector under vacuum with a filter unit that divides the collection container into a lower collection space for the fluid, a center collection space for the tissue cells and an upper vacuum space, wherein the lower collection space for the fluid and the upper vacuum space are connected with each other while bypassing the collection space for the tissue cells.

With this known device, tissue cells, in particular fatty tissue, can be suctioned from the human body and this fatty tissue can be collected in the collection container. The fatty tissue is hereby separated from the working fluid. This fatty tissue is hence available for further processing, for example for reimplantation into the human body at a different location.

For various potential applications, it would be desirable to make the fatty tissue available in very pure form, i.e., without undesirable tissue parts, such as severed and suctioned connecting tissue parts.

It is therefore an object of the invention to provide a device of the generic type, which has a simple structure and with which tissue cells, in particular fatty cells, can be separated from a biological structure and suctioned off, while undesirable tissue parts are mostly eliminated in the collected tissue cells.

This object is solved according to the invention with a device having the features of claim 1. Separation of the removed tissue cells from the fluid in a simple manner can be readily achieved by arranging a catch device for catching connecting tissue and the like from the fluid-tissue cell mixture upstream of the tissue cell collector, wherein the catch device preferably has catch structures protruding into the flow path of the fluid-tissue cell mixture. Placing the catch device upstream is important to prevent clogging of the filter device (sieve) arranged downstream of the collection space. The removed tissue cells are additionally cleaned from undesirable constituents, for example connecting tissue and the like. For example, tissue cells removed from the human body, in particular fatty tissue cells, can thus be provided with a high degree of purity.

In a preferred embodiment of the invention, the catch structures may be formed by whisker structures. In this way, connecting tissue and the like can be easily and effectively filtered from the fluid-tissue cell mixture.

In another preferred embodiment of the invention, the whisker structures are arranged in a suction line, in particular between an applicator of the device and the tissue cell collector. The overall structure of the device can the advantageously be made very compact, obviating the need for an additional component. Because the suction line is typically a component of a single-use set of the entire device, the filter device in the suction line can also be designed for single-use. The structure can then be relatively simple and optimized for the intended purpose. Cleaning is not necessary, because the filtered connecting tissue and the like can be discarded together with the single-use set.

In a preferred embodiment of the invention, the whisker structures may be made from plate-shaped bodies having planes oriented in the flow direction of the fluid-tissue cell mixture. In this way, the plate-shaped bodies can advantageously produce only a very small flow resistance and simultaneously let the tissue cells to be collected pass through. The connecting tissue and the like which have a larger structure are trapped on the plate-shaped bodies and are thus filtered out.

In another preferred embodiment of the invention, the plate-shaped bodies are radially inwardly tapered and, in particular, extend up to an imaginary centerline of the suction line. This advantageously leaves a sufficiently large flow cross section for the fluid-tissue cell mixture to pass through; advantageously, the plate-shaped bodies have a radially inwardly protruding hook-shaped structure, which enables particularly good filtering of connecting tissue and the like from the fluid-tissue cell mixture.

In another preferred embodiment of the invention, at least two groups of plate-shaped bodies are arranged sequentially in the flow direction, whereby in particular the groups of the plate-shaped bodies are arranged coaxially with a mutual offset. This design produces a particularly effective filter device which, on one hand, ensures insignificant interference with the flow cross-section of the suction line and, on the other hand, covers the entire cross-section of the suction line due to the mutual offset between groups of the plate-shaped bodies, so that the connecting tissues and the like can be particularly effectively filtered.

According to another preferred embodiment of the invention, the catch structures are oriented from the major axis of the catch device in form of rays from the center outwardly toward the wall, wherein the catch structures are preferably arranged in consecutive rows and more preferably helically twisted. A particularly effective catch structure can thus be constructed, with which connecting tissues and the like can be very effectively filtered from the fluid-tissue cell mixture, without clogging. Preferably, the catch structures may also be formed from a plurality of elongated, in particular strand-shaped articles arranged like a brush.

In this way, the tissue cells and the like can advantageously be filtered commensurate with the prevailing flow velocity of the fluid-tissue cell mixture.

Additional preferred embodiments of the invention include features recited in the other dependent claims.

Exemplary embodiments of the invention will now be described in more detail with reference to the appended drawings. These show in:

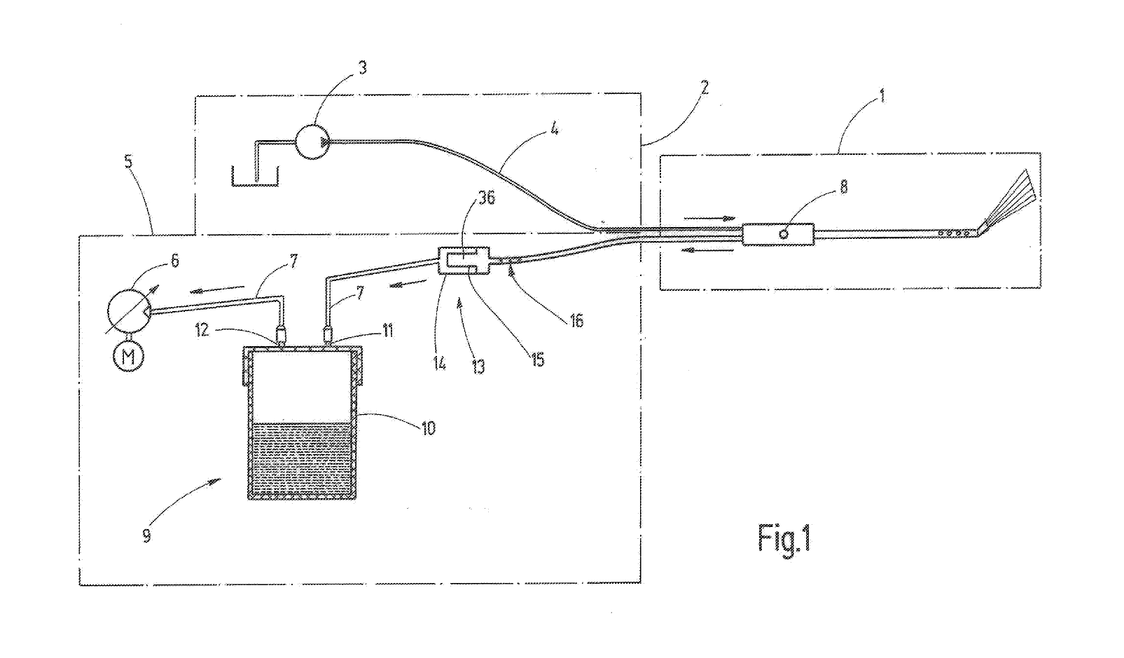

FIG. 1 a schematic diagram of a device for fluid jet separation with an integrated device for separating tissue cells from a fluid;

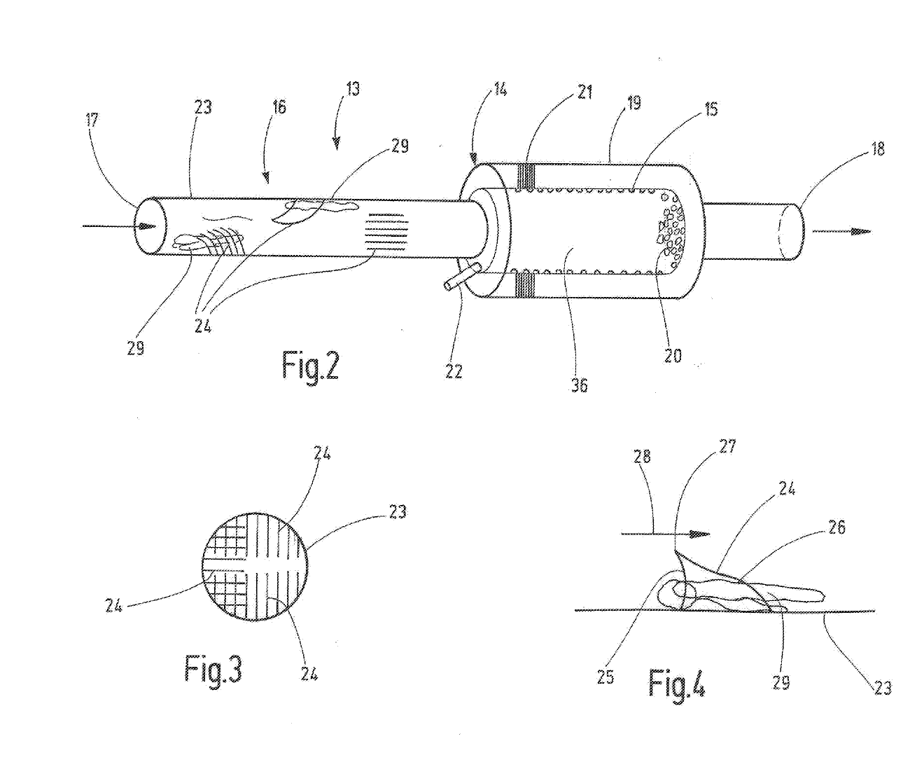

FIG. 2 a schematic cross-sectional diagram of a tissue cell collector with an upstream filter device;

FIG. 3 a schematic cross-sectional diagram through a housing section in the region of the catch device;

FIG. 4 an enlarged diagram of a plate-shaped body of the catch device;

FIG. 5 a schematic cross-sectional diagram through a tissue cell collector with an upstream catch device in a second exemplary embodiment; and

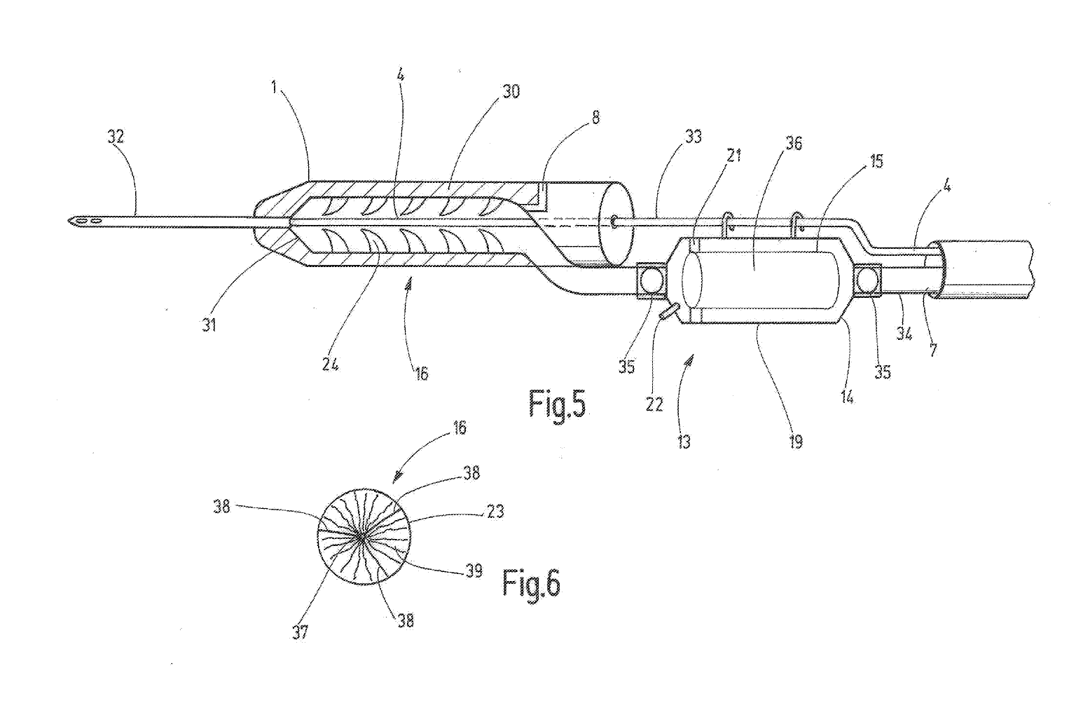

FIG. 6 a schematic cross-sectional diagram through a housing section of the catch device.

FIG. 1 shows a device for fluid jet separation with an applicator 1, which can be manually operated by an operator, for water jet-supported separation and suctioning of tissue cells from a biological structure. The device includes a pressure jet device 2 with a pressure generator 3 and a pressure line 4 for supplying the applicator 1 with a defined fluid jet. The device further includes a suction device 5 for catching the separated tissue parts (tissue cells) and the used working fluid and the autologous fluid from the biological structure. The suction device 5 includes a vacuum generator 6 and a suction line 7, wherein the suction line 7 contiguously connects the vacuum generator 6 with the applicator 1. The suction line 7 has in the region of the applicator 1 a closable bypass 8 which connects the suction line 7 to atmosphere. A residual fluid collector 9 for the suctioned fluid with a closable catch container 10 and an inlet fitting 11 and an outlet fitting 12 for the suction line 7 is located in the suction line 7. The residual fluid collector 9 for the filtered fluid is arranged in the suction direction before the vacuum generator 6. A tissue cell collector 13 is connected in the suction line 7 between the residual fluid collector 9 and the applicator 1. The tissue cell collector 13 includes a collection container 14, in which a sieve 15 is arranged which delimits a collection space 36. A catch device 16 is arranged upstream of the tissue cell collector 13.

The device for fluid jet separation illustrated in FIG. 1 has the following function:

When using the fluid jet separation method, a defined fluid separation jet exits from the applicator 1, with the effect of the fluid jet being determined by the fluid pressure generated in the pressure jet device and the structural design of the applicator 1. This effect is intended to gently separate tissue cells from a biological structure. The separated tissue cells are suctioned together with the injected working fluid and additional autologous fluids by a vacuum produced in the suction device 5. This process is frequency used in liposuction. If tissue cells suctioned in this way are to be supplied for reuse, these tissue cells are always separated from the fluid-tissue cell mixture. This is performed by the tissue cell collector 13.

In a standby position, the operator holds the closable bypass 8 in the open position, so that no suction occurs, and instead only atmospheric air is suctioned in and transported through the tissue cell collector 13. The air in the tissue cell collector 13 passes the collection space 36 through the catch device 16 and the sieve 15 in the direction toward the vacuum generator 6.

In an operating position, the closable bypass 8 is closed by the operator, so that the suction force from the vacuum generator 6 is transferred to the surgical field. The separated tissue parts (tissue cells) and the various fluids are captured and transported to the tissue cell collector 13. This mixture of tissue cells and fluid reaches the collection space 36 with the downstream sieve 15 in the tissue cell collector 13. The tissue cells are filtered with the sieve 15, whereas the fluid passes through the sieve 15 and reaches the residual fluid collector 9.

The catch device 16 ensures that undesirable tissue parts entrained in the liquid-tissue cell mixture, in particular connecting tissue and the like, are caught and prevented from traveling together into the collection space 36 to the sieve 15 of the tissue collection container 13. The tissue cells (in particular fat cells) collected in the tissue collection container 13 are therefore effectively free from undesirable tissue parts and fluid. The collected tissue cells can then be removed from the tissue cell collector 13/collection space 36 and transported onward for further processing. This may include, for example, reinjection into the same biological structure from which the tissue cells were removed.

FIG. 2 shows a schematic cross-sectional diagram of the tissue cell collector 13. The tissue cell collector 13 has an inlet 17 connecting the tissue cell collector 13 with the applicator 1. In addition, an outlet 18 is provided which connects the tissue cell collector 13 with the residual fluid collector 9. The tissue cell collector can be incorporated in the suction line 7 by way of quick-action couplings, plug-in connections and the like, which are not illustrated in detail. Importantly, the flow path for the fluid-tissue cell mixture suction between the applicator 1 towards the tissue cell collector 13 and then onward towards the residual fluid collector 9 must be ensured. The collection container 14 has an expansion 19 in which the sieve 15 is arranged. The sieve 15 is formed, for example, by a fine sieve. The fine sieve may be constructed of plastic, stainless steel and the like. The mesh size of the sieve 15 is adjusted such that the tissue cells 20, in particular fat cells, from the incoming tissue cell-fluid mixture are held back by the sieve 15, while the liquid can flow via the outlet 18 to the residual fluid collector 9. The sieve 15 is held in place in the expansion/annular gap 19 of the housing of the collection container 14 by a clamping device/attachment means 21 and the like and forms the boundary for the collection space 36. The clamping device/attachment means 21 can completely or partially close off the expansion/annular gap 19. Partial closure allows the fluid cell mixture to overflow into the expansion/annular gap 19 and onward to the outlet 18, if the collection space 36 is full, without having to pass through the sieve 15. A main flow direction can be optimally set by selecting/sizing the cross-sectional area of the partial closure of the expansion/annular gap 19 in relation to the size of the sieve 15. The main flow direction preferably extends through the collection space 36 and the sieve 15. The main flow direction only switches via the partially closed expansion/annular gap 19 to the outlet 18 when the collection space 36 is full. The collection container 14 also includes an indicated closable withdrawal opening 22 through which the tissue cells 20 collected in the collection space 36 can be withdrawn with a suitable device.

The tissue cell collector 13 also includes the catch device 16 which is arranged inside a housing segment 23. The catch device 16 includes plate-shape bodies 24 which are arranged in groups—here three groups—on the interior wall of the housing segment 23. The plate-shaped bodies 24 each extend radially inwardly into the housing segment 23. This radial extent terminates approximately at an imaginary center line of the housing segment 23. Each group of the bodies of the plate-shaped bodies 24 has several, mutually parallel plate-shaped bodies 24, with planes that extend in the flow direction of the fluid-tissue cell mixture. In this way, the plate-shape bodies 24 form a so-called whisker structure configured to filter the fluid tissue cell mixture entering the tissue cell collector 13. The three groups of plate-shaped bodies 24 are here consecutively arranged in the flow direction. The plate-shaped bodies 24 also each extend coaxially with an offset from the interior wall of the housing segment 23 in a direction toward the imaginary centerline. According to the diagram of FIG. 2, first a first group of the plate-shaped bodies 24 extends downward into the interior space of the housing segment 23, then a next group of the plate-shape bodies 24 extends into the interior space of the housing segment 23 from above (offset by 180°) and finally a third group of the plate-shaped bodies 24 extends laterally into the interior space of the housing segment 23 (offset by 90°).

However, only two groups of the plate-shaped bodies 24 or more than three groups of the plate-shaped bodies 24 may be provided, which is not shown in the exemplary embodiment. The plate-shape bodies 24 may also be arranged at the same height and mesh with each other in a comb-like structure.

FIG. 3 shows schematically a cross-sectional diagram through the housing segment 23, as viewed from the inlet 17 into the housing segment 23. The arrangement of the plate-shaped bodies 24 is clearly visible. The first group of the plate-shape bodies 24 is here arranged at the bottom, the second group of the plate-shape bodies 24 at the top and the third group of the plate-shape bodies 24 on the left side (corresponding to the diagram of FIG. 3). This creates a superpositioned lattice structure, wherein the plate-shaped bodies 24 of the filter device 16 only insignificantly decrease the flow cross-section inside the housing segment 23.

FIG. 4 shows schematically an enlarged diagram of a plate-shaped body 24 extending from the interior wall of the housing segment 23. The diagram in FIG. 4 clearly shows that the individual plate-shaped body 24 is radially inwardly tapered and a first flank 25 and a second flank 26 are joined to a hook-shaped tip 27. The first flank 25 is formed concave against the flow direction 28, wherein the second flank 26 is convex in the flow direction 28. This illustration is only exemplary. Other shapes of the catch structures are feasible, for example oriented from the main axis of the catch device 16 in form of rays from the center outwardly towards the wall.

The diagrams in FIGS. 2, 3 and 4 clearly show that the catch device 16 is able to catch undesirable tissue parts, in particular connecting tissue and the like, from the fluid-tissue cell mixture before entering the collection space 36. FIGS. 2 and 4 show relatively large connecting tissue parts 29, which have been caught/got stuck in the whisker-like structures of the plate-shaped bodies 24. Only the previously cleaned fluid-tissue cell mixture then reaches the collection space 36 with the sieve 15, so that the tissue cells 20 filtered by the sieve 15 no longer contain undesirable tissue parts.

FIG. 5 shows a second modified embodiment of the invention, wherein identical parts have identical reference symbols as those in the previous Figures—in spite of their sometimes different arrangement.

In the modified embodiment illustrated in FIG. 5, the catch device 16 is integrated in a handle 30 of the applicator 1. The handle 30 has herein a suitably constructed channel 31 in which the plate-shape bodies 24 are arranged. The channel 31 is connected with an actual surgical instrument 32, which is used to supply the working fluid (not illustrated here) and to suction the fluid-tissue cell mixture in a conventional manner.

The suction line 7 is connected via a segment 34 with the collection container 14, in which—as mentioned above—the sieve 15 is arranged.

The reference symbol 33 indicates a pressure line for supplying the working fluid.

The tissue cell collector 13 is releasably incorporated in the segment 34 by way of illustrated quick-action couplings 35. In other words, the tissue cell collector 13 is configured for exchange. It thereby becomes feasible to combine the applicator 1 with optionally differently constructed tissue cell collectors 13. Alternatively, a continuous suction or line segment may be used instead of the tissue cell collector 13, so that the applicator 1 can also be used when tissue is not being collected. The applicator 1 itself is typically a single-use part, i.e., it is discarded after its intended use. The existence of the catch device 16 does therefore not represent an additional disadvantage even if no tissue is to be collected.

The device illustrated in FIG. 5 has the following function:

In a standby position, the closable bypass 8 is open. The vacuum of the vacuum generator 6 is then connected to atmosphere via the segment 34 and the tissue cell collector 13, the quick-action coupling 35 and the bypass 8. When the bypass 8 is closed by the operator, vacuum is applied to the surgical instrument 32 via the segment 34 and the tissue cell collector 13. The fluid-tissue cell mixture which is suctioned via the surgical instrument 32 then moves through the catch device 16 and the collection container 14 into the collection space 36. Only the residual fluid reaches the residual fluid corrector 9 (FIG. 1) through the sieve 15.

FIG. 6 shows schematically a cross-sectional diagram through the housing section 23 of a catch device 16. In this embodiment, a central retaining body 37 is provided which extends coaxially to the housing section 23. The retaining body 37 is, for example, connected with the housing section 23 by illustrated ribs 38. Catch structures extend from the retaining body 37 toward the wall of the housing section 23. The catch structures are formed by strand-shaped bodies which are arranged inside the housing section 23 like a brush. The fluid-tissue cell mixture then flows through these structures, allowing the tissue cells and the fluid to pass through, whereas the connecting tissue parts 29 get caught in the catch structures and therefore cannot reach the collection space 36 or the sieve 15, respectively. The strand-shaped bodies 39 can be arranged on the retaining body 37 in several spaced-apart rows or have a helical arrangement.

### LIST OF REFERENCES SYMBOLS

1 Applicator
2 Pressure jet device
3 Pressure generator
4 Pressure line
5 Suction device
6 Vacuum generator
7 Suction line
8 Bypass
9 Residual fluid collector
10 Catch container
11 Inlet fitting
12 Outlet fitting
13 Tissue cell collector
14 Collection container
15 Sieve
16 Catch device
17 Inlet
18 Outlet
19 Expansion/annular gap
20 Tissue cells
21 Clamping device/attachment means
22 Withdrawal opening
23 Housing section
24 Plate-shaped body
25 First flank
26 Second flank
27 Hook-shaped tip
28 Flow direction
29 Connecting tissue parts
30 Handle
31 Channel
32 Surgical instrument/suction tube with coaxial pressure line
33 Segment/pressure hose
34 Segment/vacuum hose
35 Quick-action coupling
36 Collection space
37 Central retaining body
38 Ribs
39 Strand-shaped body

1. Device for fluid jet-supported separation and suctioning of tissue cells from a biological structure, with a pressure generator for supplying a defined fluid jet to an applicator of the device, with a suction device for removing the separated tissue cells and the used fluid from the biological structure, wherein a suction device comprises a vacuum source and a suction line for connecting the applicator with the vacuum source, wherein a tissue cell collector is incorporated in the suction line, characterized in that a catch device (16) for catching connecting tissue and the like from the fluid-tissue cell mixture is arranged upstream of the tissue cell collector (13).

2. Device according to claim 1, characterized in that the catch device (16) comprises catch structures which protrude into the flow path of the fluid-tissue cell mixture.

3. Device according to claim 2, characterized in that the catch structures are formed by whisker structures.

4. Device according to claim 3, characterized in that the whisker structures are arranged in the suction line (7).

5. Device according to one of the preceding claims, characterized in that the whisker structures are formed by plate-shaped bodies (24) having planes which are oriented in a flow direction of the fluid-tissue cell mixture.

6. Device according to one of the preceding claims, characterized in that the plate-shaped bodies (24) are tapered radially inwardly.

7. Device according to one of the preceding claims, characterized in that the plate-shaped bodies (24) extend to an imaginary centerline of the suction line.

8. Device according to one of the preceding claims, characterized in that at least two groups of plate-shaped bodies (24) are arranged consecutively in the flow direction.

9. Device according to one of the preceding claims, characterized in that the groups of the plate-shaped bodies (24) are arranged coaxially with a mutual offset.

10. Device according to one of the preceding claims, characterized in that the catch structures are oriented from the major axis of the catch device (16) in form of rays from the center outwardly to the wall and arranged in sequential rows, preferably with a helical twist.

11. Device according to claim 10, characterized in that the catch structures are formed by brushes.
